(19) United States
(12) Patent Application Publication
(10) Pub. No.: US 2022/0211239 A1
(43) Pub. Date: Jul. 7, 2022

(54) DEVICE FOR CLEANING WALKABLE SURFACES

(71) Applicant: TECHNOLOGICAL SYSTEMS BY MORO S.R.L., Fiume Veneto (PN) (IT)

(72) Inventors: Gianpietro MORO, San Quirino (IT); Andrea MORO, Pordenone (IT)

(73) Assignee: TECHNOLOGICAL SYSTEMS BY MORO S.R.L., Fiume Veneto (PN) (IT)

(21) Appl. No.: 17/612,969

(22) PCT Filed: May 21, 2019
(86) PCT No.: PCT/IT2019/000039

§ 371 Date: Nov. 19, 2021

(51) Int. Cl. A47L 11/30; A47L 11/40; A61L 2/10

(52) U.S. Cl. CPC A47L 11/305; A61L 2202/14; A47L 11/4069; A47L 11/4083; A47L 11/4088; A47L 11/4016; A47L 11/4075; A47L 11/4008; A47L 11/4011; A47L 11/405; A47L 11/4072; A47L 11/4066; A61L 2/10; A61L 2202/11; A61L 2202/17; A47L 11/4061

(57) ABSTRACT

A scrubber-dryer device for cleaning walkable surfaces, as for example floors, pavements, asphalted roads, etc., adapted to move forward automatically and to be steered manually by an operator, and including a cleaning lower base body connected on its upper part by a movable jointing element to a steering means lengthened upward the lower base body including a frame to which one or more lower brushing means are fixed to, at least a wetting system fixed to the frame, and at least a dirt collection system fixed to the frame, the jointing element formed by a lengthened elastic and internally hollow push element, constrained with its lower end portion to the frame, in the barycenter point of the lower body and with its upper end portion to the steering element.

The invention relates to a device for cleaning walkable surfaces, adapted to move forward automatically and to be steered manually by an operator for cleaning walkable surfaces, as for example floors, roads, pavements, etc., particularly where there are present tight spaces for operating such a device or spaces that are difficult to be reached.

To date we know different types of machines for cleaning floors by means of motorized rotating brushes, which are also wet by means of a relative delivery system for liquids.

Particularly, in the technical field of the present invention, it is known a first type of floor scrubber machine constituted by a lower support frame leant on wheels and one or more motorized brushes with vertical rotation axis or slightly inclined, adapted to rub the floors to be cleaned and to move forward automatically the cleaning machine thanks to the friction with the floor, without the operator must push it or without the use of motorized wheels.

Such a frame substantially supports on its upper part the unit motor-battery for actuating such cleaning brushes, the tanks for the clean water, for the possible detergent, and for the dirty water, sucked by an electrical suction system, which is placed on the upper part of said frame too and is associated with an element, which is lengthened horizontally and made of rubber, positioned lowerly in the back part of said support frame, and adapted to collect the dirty water resulted from the brushing action of the floor by the wetted rotating brushes.

A fixed guiding body is also secured on the support frame, which is lengthened upward and ending with a handle associated with a console equipped with commands adapted to be grasped by the operator for guiding the cleaning machine and for commanding the electrical and electronic parts thereof.

The component elements supported on the upper part of the support frame and to the lower part of the guiding means are covered by a shaped casing.

Such a type of cleaning machine for floors, also if it is easily to be guided and commanded, is not adapted to clean floors and tight spaces or in spaces wherein objects are placed on the top and spaced away from same, as for example hanging furniture, tables, chairs, etc., because the machine has considerable dimensions.

In this technical field, it is also known a second type of machine for a wet cleaning of floors by means of rotating brushes, constituted by a base body connected, by means of articulated jointing element, to an upper lengthened guiding means.

Said base body comprises at its lower part one or more motorized rotating elements, as for example brushes, rollers with bristles, rollers with sponge, etc., with a rotation axis that is horizontal, vertical or inclined, adapted to scrub the floors for cleaning them.

An element, made of rubber, is positioned behind said motorized rotating elements, with respect to the direction of advancement of the machine, and is adapted to receive and to keep the dirty brushed from the floor, that is then sucked by means of a suction system and collected in a suitable tank. It may be present also a wetting system of said motorized brushes or similar, for increasing the cleaning efficiency thereof.

On the guiding means, in addition to the handle for guiding the machine, there are present the command for setting the operational functions of the same machine.

For facilitating the operator, such rotating elements may move forward the machine during the brushing, thanks to the friction with the floor.

Various component elements, including the motors, the batteries, the tanks, the operation electrical and electronic systems, the elements of the wetting and suction systems are applied and arranged between the upper part of the base body and the guiding means, in particular with at least tank applied on this latter.

This second type of known cleaning machines use a articulated jointing element between the guiding means and the base body, in a manner to be able to bring the upper end portion of the same guiding means nearer to the floor, so that it is possible to clean portions of floors that are covered in a spaced manner by tables, chairs, hanging furniture, etc.

As articulated jointing elements there are used joints with one rotation axis, for example that allow a tilting movement of the guiding means, or joints with two rotation axis orthogonally to each other and placed on two different levels, as for example a universal joint, that allow to the guiding means to be bent with its upper end portion toward the floor in different direction, and by twisting the same guiding means, they allow that the base body may rotate toward right or left.

In this second type of cleaning machine, the lengthened guiding means may be inclined in a limited manner with respect to the base, up to it goes into contact with it, and this may be result a limitation for cleaning areas of the floor with objects positioned above it and spaced superiorly, and that have a considerable depth.

Furthermore, the base may not rotate on itself on a vertical axis without inclining on a horizontal axis, except with the guiding means positioned vertically, and that in narrow spaces, wherein it is necessary to incline the same guiding means, compels the operator to pull the cleaning machine in the reverse direction with respect to the one in which the motorized rotating elements create propulsion, therefore by tiring the operator and by straining the motors of said rotating elements. This type of cleaning machine is not provided with dampening means that damp collisions with external elements, places on the floor to be treated, thereby creating the risk of damages to the same machine.

By placing the tanks for clean water and/or dirty water on the guiding means, in addition to the weight of the liquid contained in the tanks, the water, passing from a tank to the other one, changes the weight distribution on the guiding means, thereby causing a change of the operator's posture. The object of the present invention is to delete the above said problems and limits, and additional advantages will be described following.

This aim and other ones are obtained according to the invention by means of the use of a device for cleaning walkable surfaces realized in a different manner with respect to the known solutions from the prior art, with the characteristics and operating as it will be described, by way of a not-limitative example only and with reference to the accompanying drawings, in which:

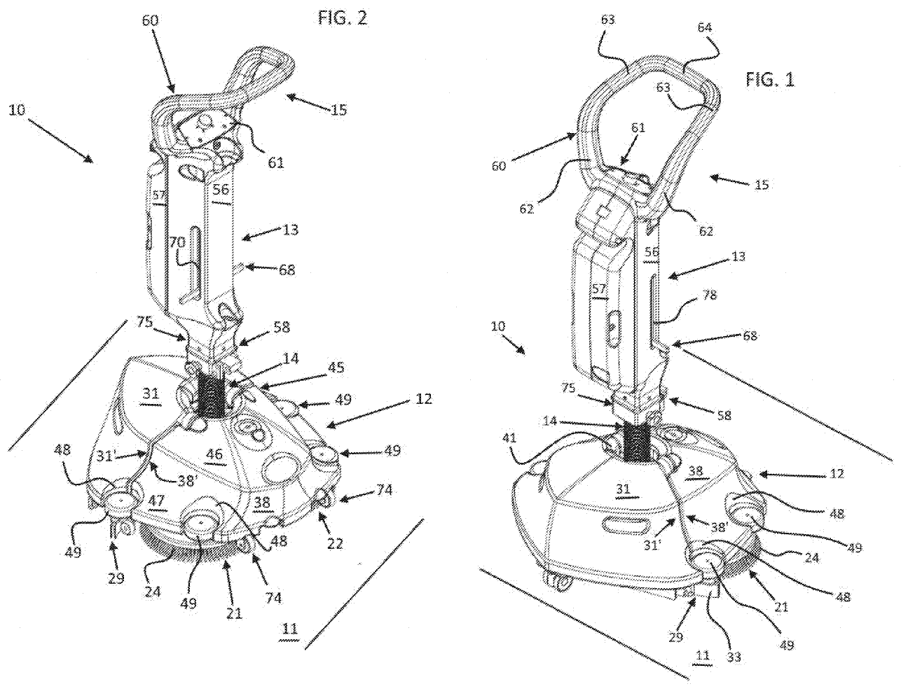

FIG. 1 shows a front-side perspective view of a first example of the device for cleaning walkable surfaces in a starting rest position thereof, according to the invention;

FIG. 2 shows a back-side perspective view of the device of FIG. 1;

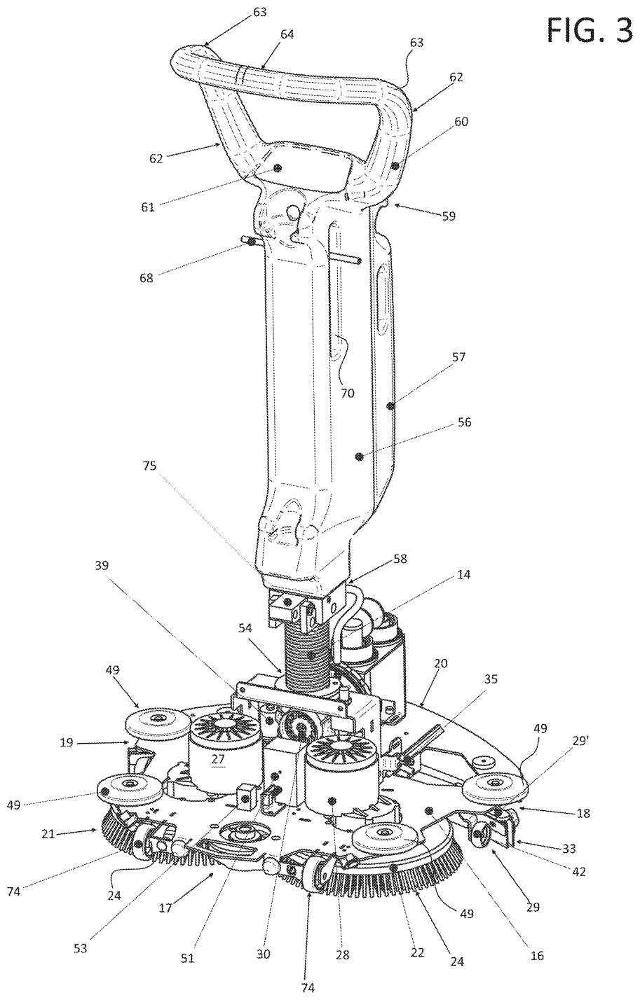

FIG. 3 shows a back perspective view of the cleaning device of FIG. 1 in an operational position thereof, without some of component elements thereof for giving prominence additional internal component elements thereof;

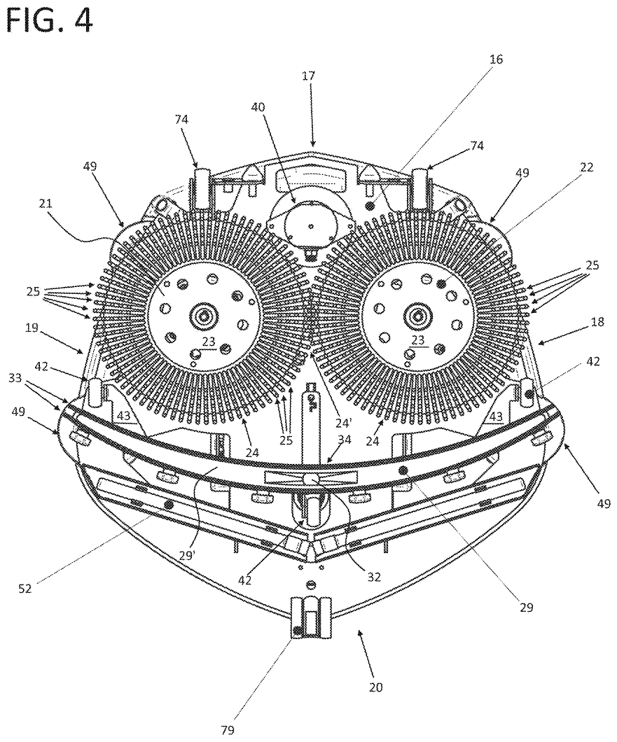

FIG. 4 shows a view from below of the cleaning device of FIG. 1;

FIG. 5 shows a side view of a group of elements that is shown in FIG. 3, in a working position thereof;

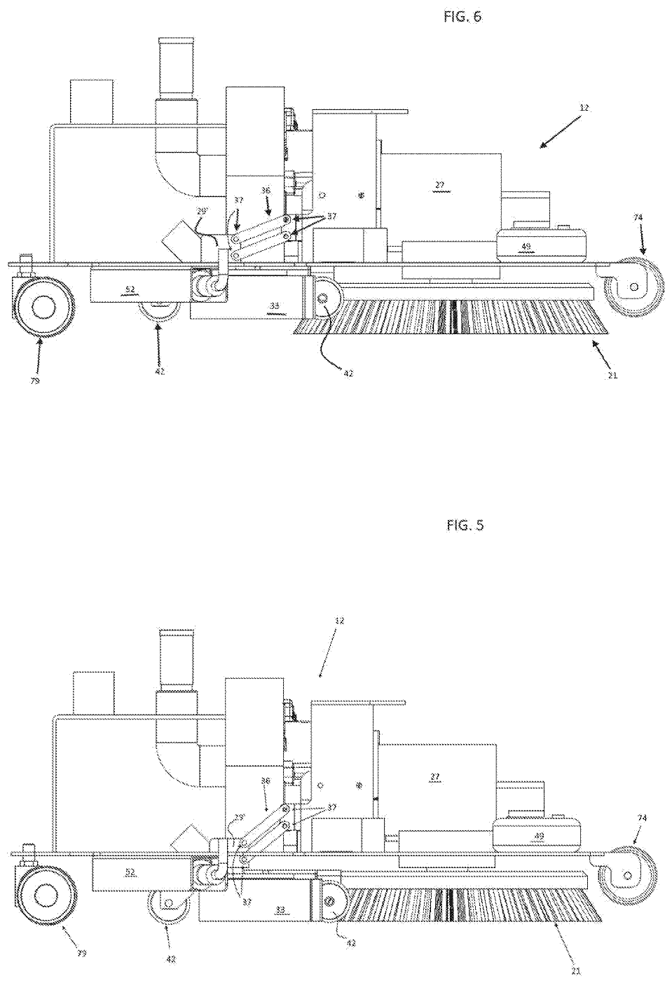

FIG. 6 shows a side view of the group of elements of FIG. 5, in a not-working position thereof;

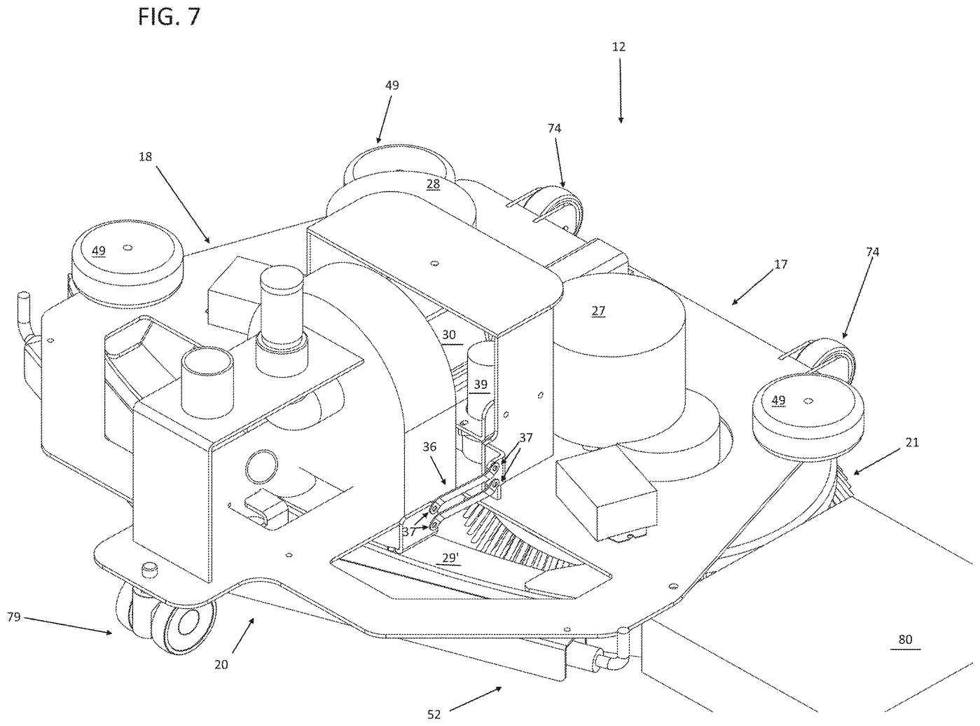

FIG. 7 shows a back-side perspective view of the group of elements shown in FIG. 3, in an operational position thereof, and one external element;

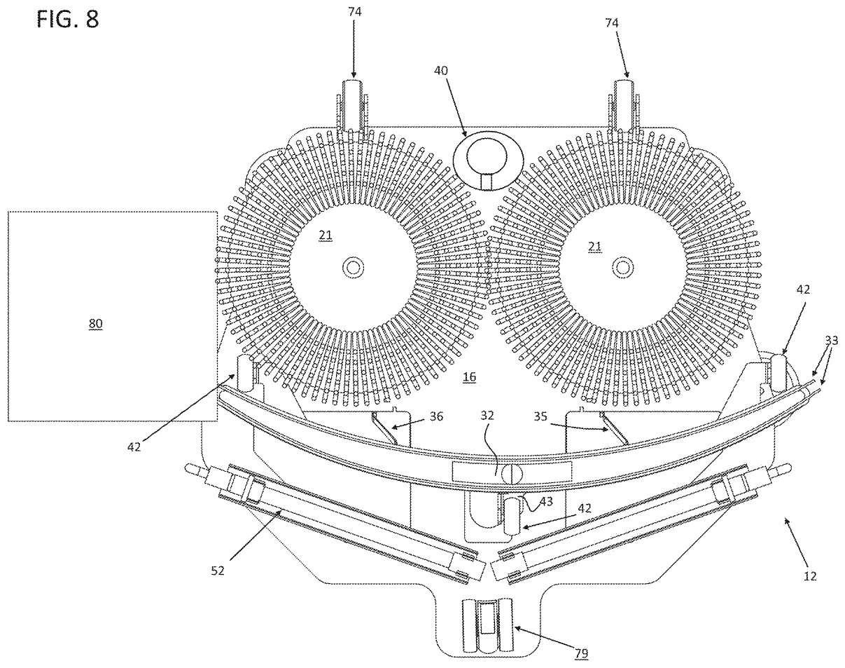

FIG. 8 shows a view from below of FIG. 7;

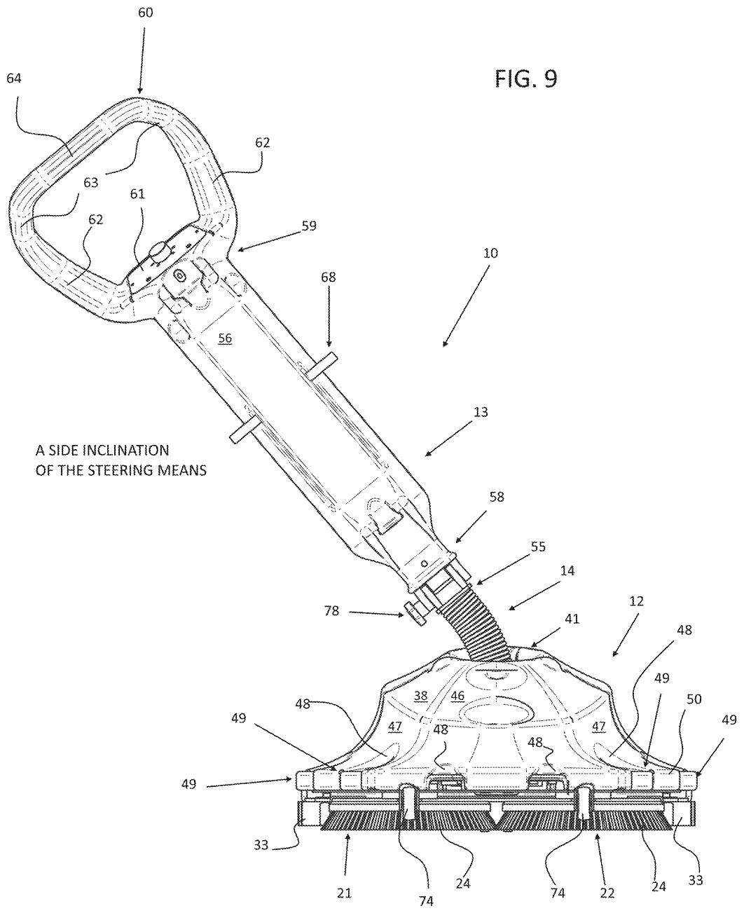

FIG. 9 shows a front view of the cleaning device in an operational position thereof;

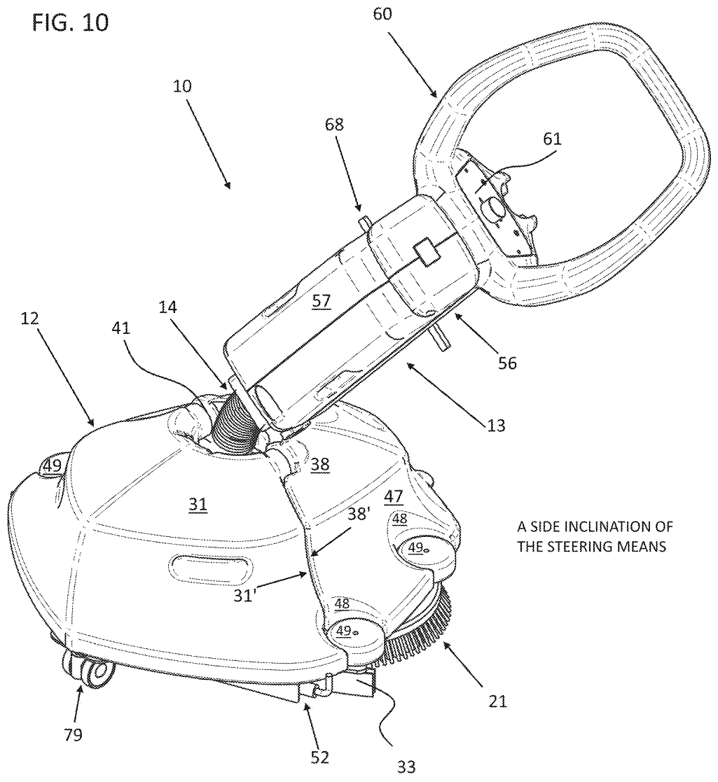

FIG. 10 shows a back-side perspective view of the cleaning device shown in FIG. 9;

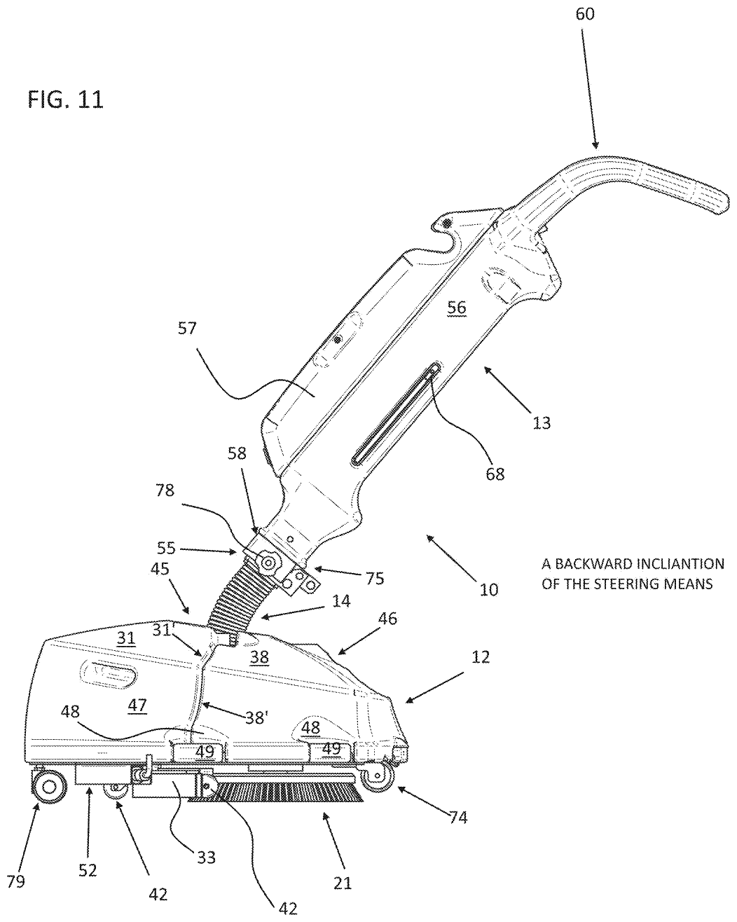

FIG. 11 shows a side view of the cleaning device in an additional operational position thereof;

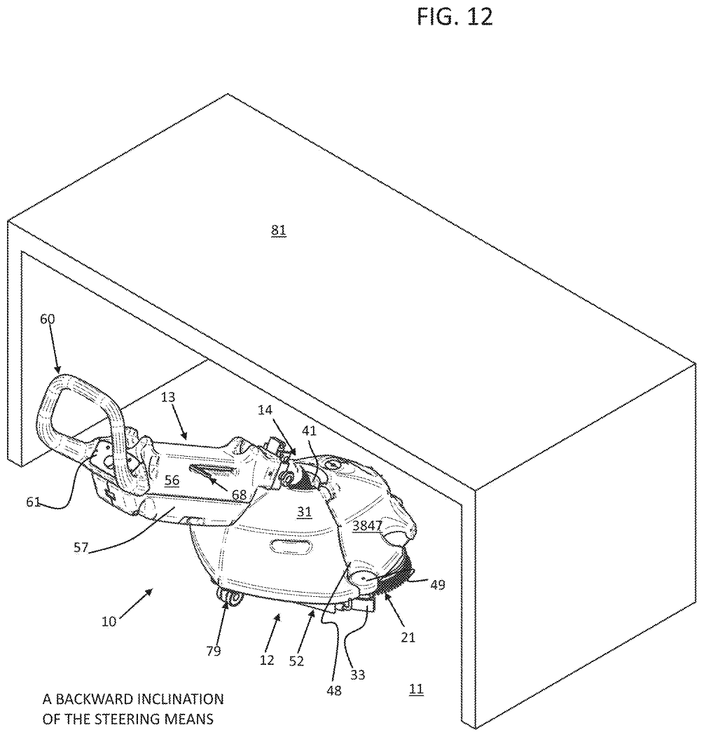

FIG. 12 shows a back-side perspective view of the device of FIG. 11, together one external element;

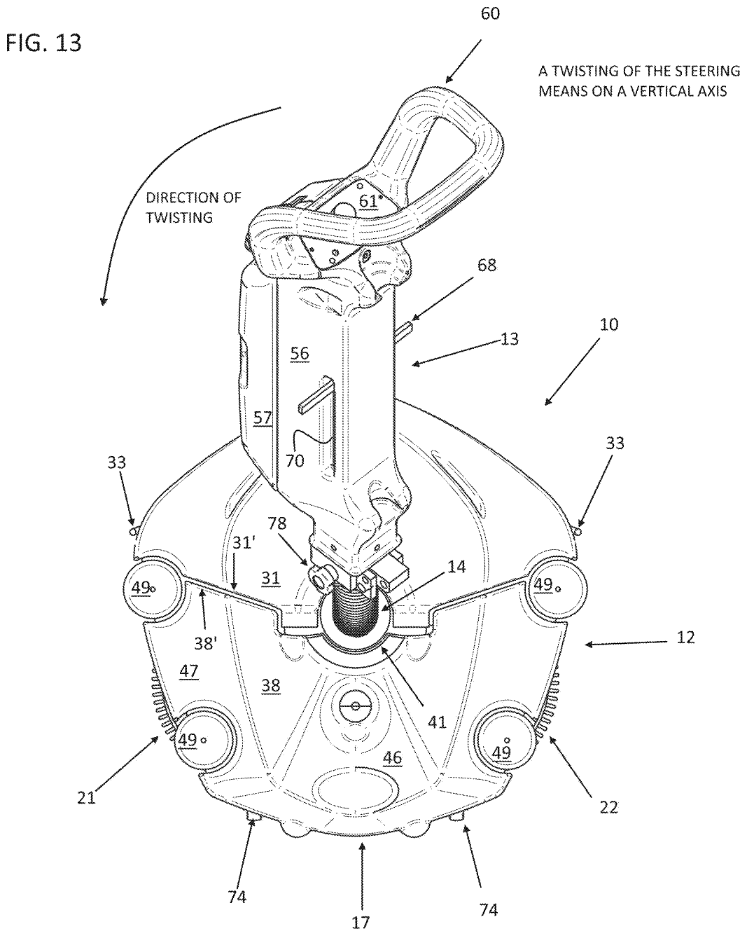

FIG. 13 shows a perspective view from the top of the cleaning device, in an additional operational position thereof;

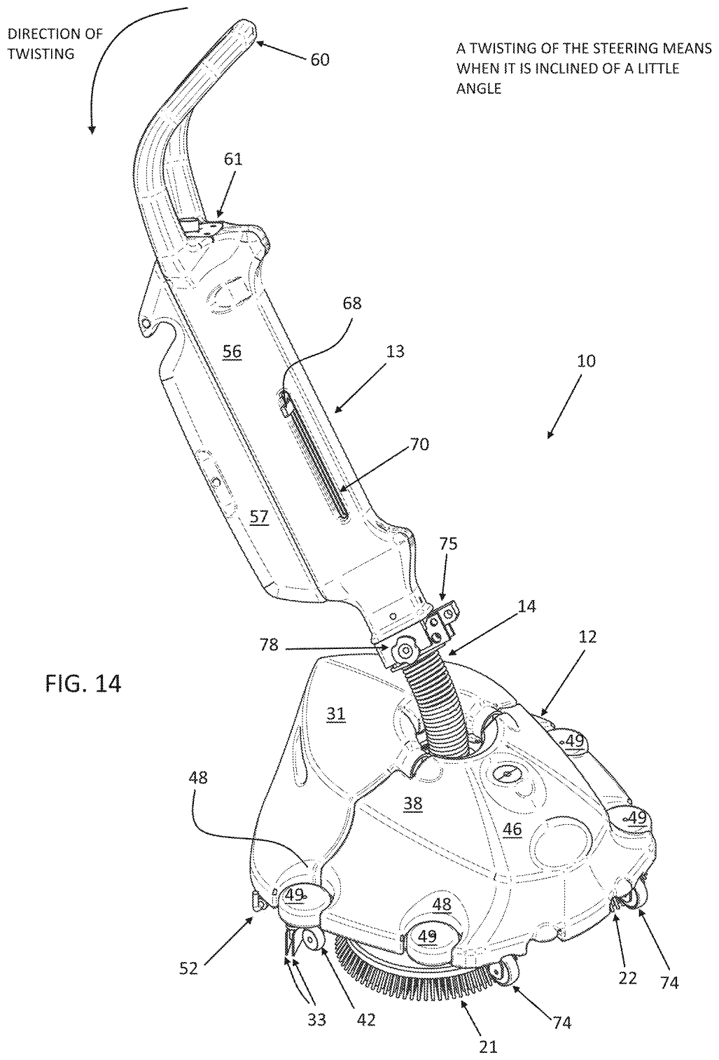

FIG. 14 shows a front-side view of the cleaning device, in an additional operational position thereof;

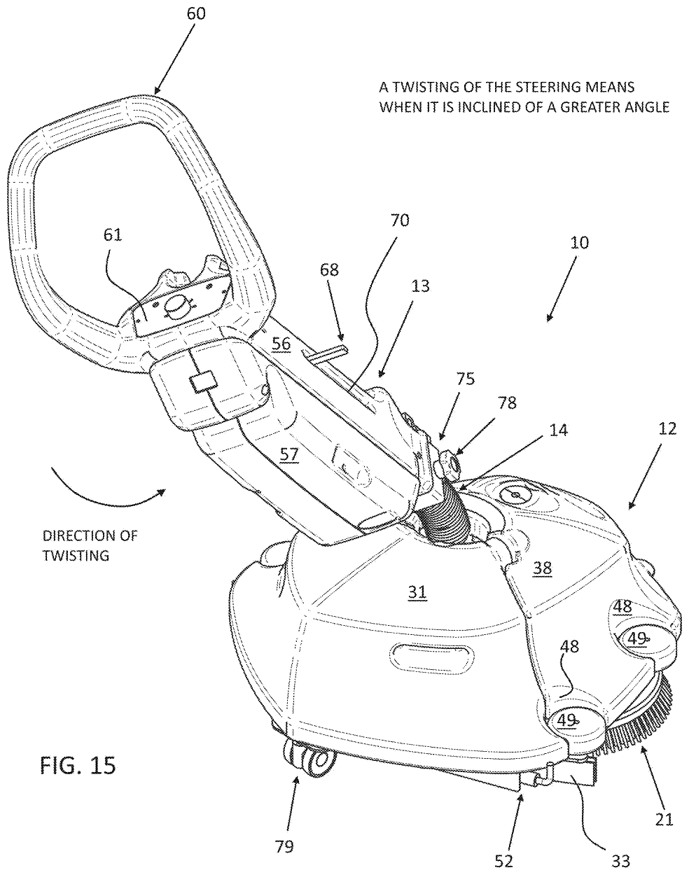

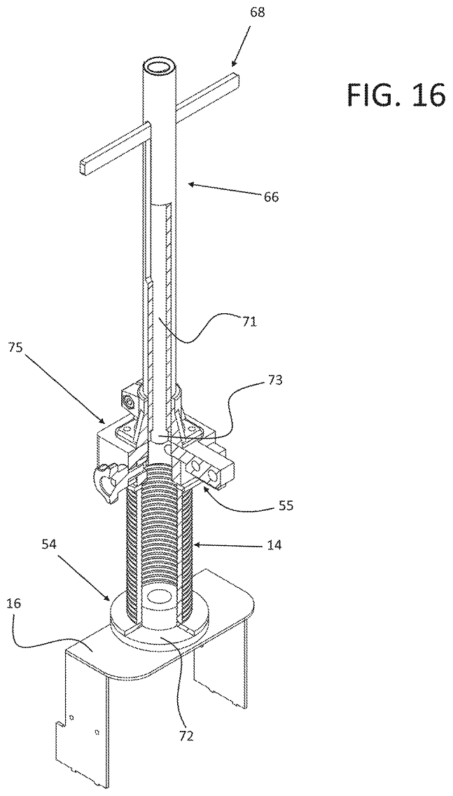

FIG. 15 shows a back-side view of the cleaning device, in an additional operational position thereof;

FIG. 16 shows a perspective view of a group of internal elements of the cleaning device with one element in a rest position thereof;

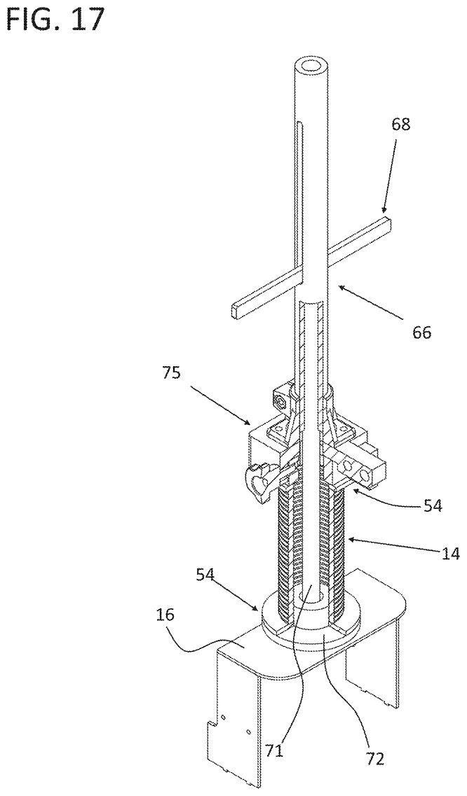

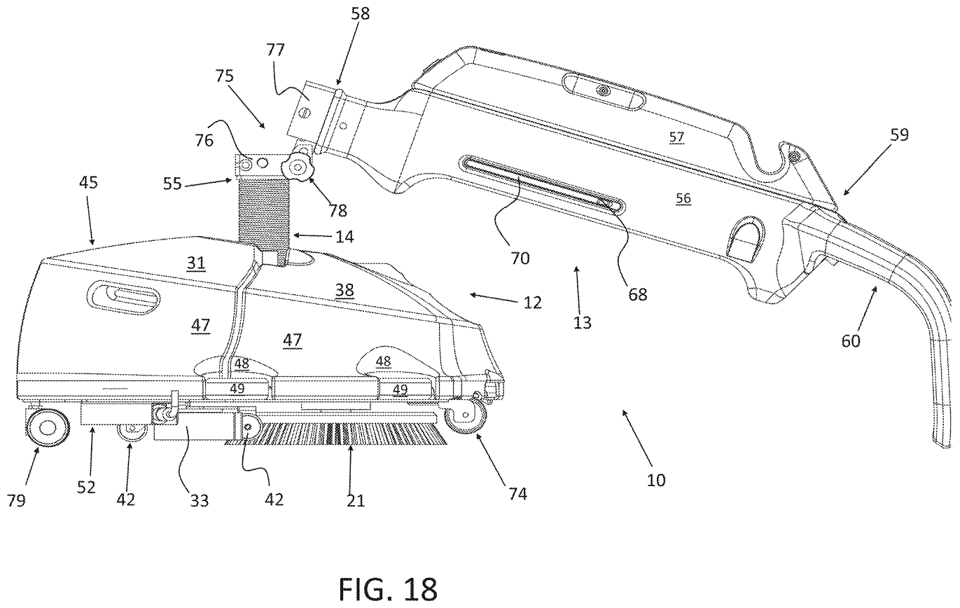

FIG. 17 shows a perspective view of the group of internal elements of FIG. 16, with the above said element in a operational position thereof;

FIG. 18 shows a side view of the cleaning device in a storage/packaging position thereof;

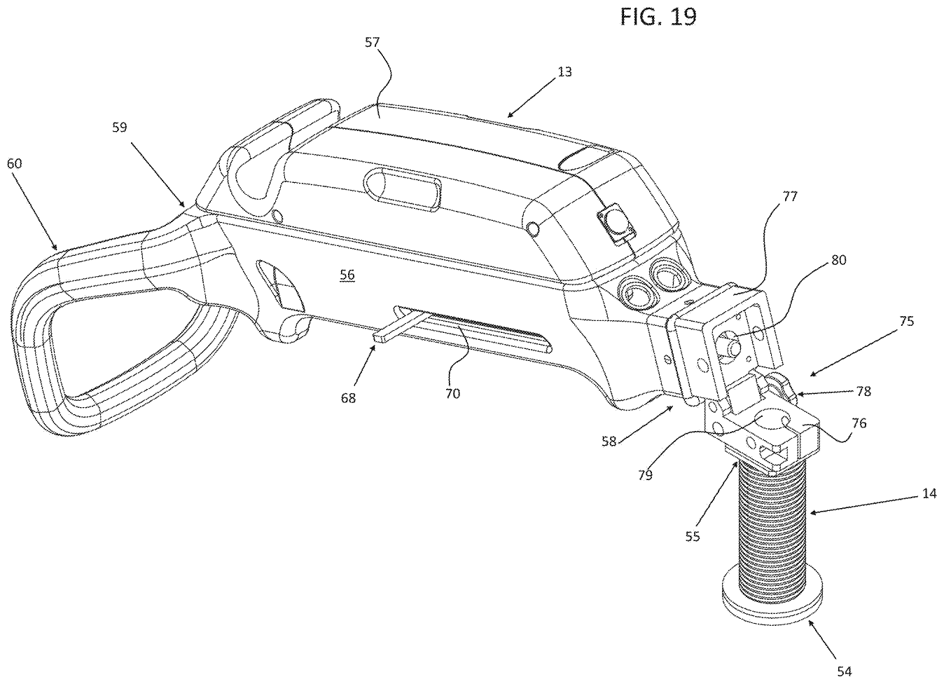

FIG. 19 shows a side perspective view of the group of elements of FIG. 18;

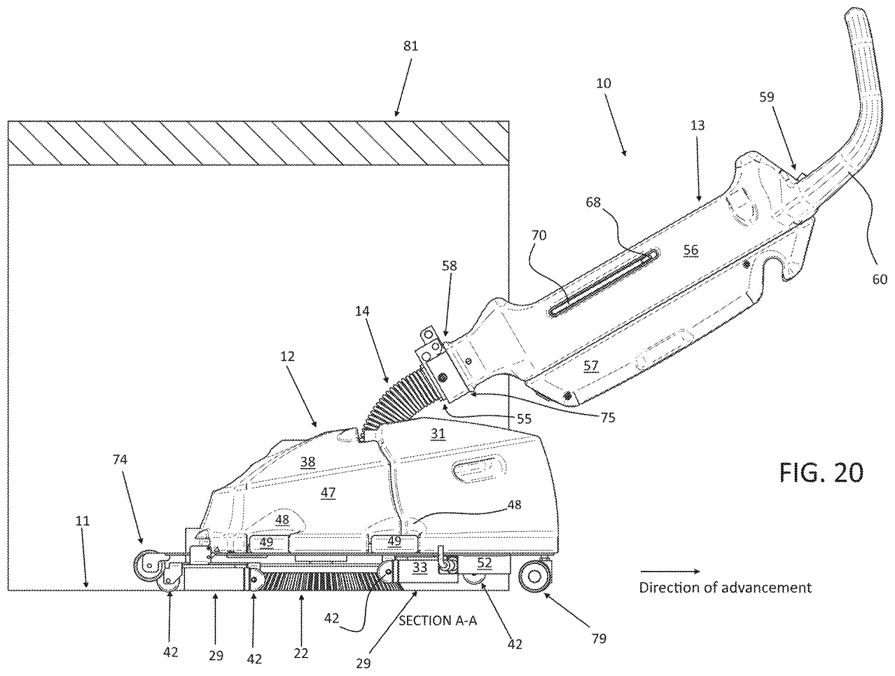

FIG. 20 shows a side view of a second example of the cleaning device in a working position thereof, together one external object.

The invention relates to a device for cleaning walkable surfaces, adapted to move forward automatically and to be steered manually by an operator for cleaning walkable surfaces, as for example floors, roads, pavements, etc., particularly where there are present tight spaces for operating such a device or spaces that are difficult to be reached.

As it is visible particularly in FIGS. 1-2, the device 10 is realized as scrubber-dryer machine for cleaning walkable surfaces 11, as for example floors, pavements, asphalted roads, etc., and is substantially constituted by a cleaning lower base body connected on its upper part to a steering means lengthened upward, by means of a movable jointing element 14, which is conformed in such a way that such a lower body 12 may move forward at least in one travel direction and that said steering means 13, from one starting position thereof perpendicular with respect to the base body 12, be inclinable in all direction without that the base body 12 being inclined, up to be able to touch the walkable surface 11 with its upper free end portion 15, and may also steer said lower body 12 apart from the inclination wherein it is, as it will be described.

As it is visible particularly in FIGS. 3-4, said cleaning lower body 12 is constituted by a support frame 16 having a front outline 17, two side outlines 18, 19, and a back outline 20, and onto which there is fixed the movable jointing element 14, in such a way to be positioned in the barycenter of the same lower body 12.

At least a brushing means is housed lowerly to such a support frame 16, by means of bearings or axle boxes or equivalent means, in such a way to be rotatable along an axis slightly inclined with respect of the vertical of the device 10 leant on to a horizontal plane, thereby be able to brush the walkable surface 11 and removing the dirt present on this latter.

In the not-limitative examples described herein there are provided two brushing means 21 and 22, identical to each other, and each constituted by a raised cylindrical plate 23 provided with a plurality of bristles 24 arranged radially in the lower part of the same cylindrical plate 23 and directed outward with respect to this latter and downward.

Said two brushing means 21 and 22 are arranged side by side to each other in a transversal manner with respect the orientation of the lower body 12, in such a way that an internal portion 24′ of the bristles 24 of each means 21, 22 is intersected with its own end portions 25, whereas one external portion of the bristles leans out laterally with its own end portions 25 with respect to the outline of the lower body 12, furthermore the rotation axis of both brushing means 21 and 22, with extension upward, is slightly inclined inward the lower body 12, with the same angle, in such a way that the bristles 24 of the internal portions 24′ be more pressed against the walkable surface 11 to be cleaned.

Said brushing means 21 and 22 are actuated in rotation by means of respective electrical actuating means 27, 28, constituted as for example by electrical motors, which are arranged on the upper part of said frame 16, and above to the respective brushing means 21 and 22, that they have to actuate. Said brushing means 21 and 22 rotates in opposite directions to each other, in particular the internal portion 24′ of the bristles 24 move backward with respect to the direction of advancement of the device 10, in such a way to convey the brushed dirty in a central and back position with respect to themselves, and in such a way that, the friction made by the rotation of the brushing means 21 and 22, with an inclined rotation axis, between the internal portions 24′ of the bristles 24 and the walkable surface 11 made in turn a traction such to allow to the device 10 to move forward automatically in a rectilinear way when the brushing means 21 and 22 are actuated in rotation. At least a system for collecting the dirty conveyed by the internal portion 14′ is placed on the back and spaced away from said brushing means 21 and 22, which system is constituted by at least a restraining means 29 for the dirty, adapted to restrain the conveyed dirty, associated to at least a suction means 30, adapted to suck the restrained dirty, and at least a collection means 31, adapted to contain the sucked dirty.

In the case it is provided for a manually or automatic inversion of the rotation direction of the brushing means 21 and 22, at least a second system for collecting the dirty constituted by at least a restraining means 29 for the dirty, at least a suction means 30, and at least a collection means 31, is placed on the front part and spaced away with respect to the same brushing means, in such a way that in the case of the inversion of the rotation direction of these latter, it is always at the back with respect to them, such a second example of device 10 is represented in FIG. 20.

Said restraining means 29 for the dirty is constituted by a lengthened semi-circular support 29′ provided with at least a vertical through hole 32 and which supports on its lower edge at least a lengthened element 33, made of a semi-rigid material, as for example rubber, said support 29′ being positioned transversally with respect to the orientation of the device 10, with its concave part 34 turned toward the two brushing means 21 and 22, and extending between the two side outlines 18, 19, whereas the lengthened element 33 being extended beyond these latter.

Said support 29′ is connected to the lower part of the frame 16 by means of at least two pair of levers 35, 36, pivoted at their end portions 37 parallel to each other, and arranged longitudinally with respect to the orientation of the device 10, thereby creating a pantograph linkage system, which levers are made of elastic metal, preferably of harmonic steel, and are adapted to rotate on their end portions 37 according the vertical movement caused by the restraining means 29 that follows the conformation of the walkable surface 11, thereby keeping itself always parallel to this latter, or it may be lifted or lowered manually or automatically when the operator considers it necessary, that is when it is foreseen the inversion of the rotation direction of the brushing means 21 and 22, and said collection system of the dirty is on the front with respect to the direction of advancement of the device 10 in an operational state thereof, furthermore, such levers are structured and adapted to be bent transversally when the restraining means 29 bumps laterally against an external obstacle 80, for then returning automatically in the original position thereof after having passed such an obstacle.

Such a restraining means 29 may be lifted by rotating the levers 35, 36 in a manual way or by means of one electrical actuator means 51, placed on the upper part of the frame 16, and that operates in the manner that will be described further, said restraining means 29 being kept lifted by means of quick fixing means or magnetic means or electro-magnetic means, or equivalent.

Said restraining means 29 may be provided with two support wheels 42, placed laterally and/or centrally with respect to the lengthened support 29′ and are fixed by means of metallic brackets 43. Said suction means 30 is of the electrical type, and is arranged on the upper part of said frame 16 and above said restraining means 29, and with its inlet mouth is connected in a communicating way to said vertical through hole 32, in such a way that when it is actuated it may suck through it the dirty restrained by the same restraining means 29.

The outlet mouth of such a suction means 30 is connected in a communicating manner to said collection means 31, in such a way that the sucked dirty may be conveyed and collected inside it. Said collection means 31 is constituted by a first tank arranged in a removable manner above the back half portion of said frame 16 and the components placed in this portion of the frame, and has a base that follows the outline and the dimensions of the portion of frame on which is leant and a height that doesn't interfere with the jointing element 14.

Said brushing means 21, 22 are associated with a wetting system adapted to force the washing liquid on to the same means, for making most efficient the cleaning of the walkable surface 11, for then be mixed to the brushed material to be removed and conveyed by the bristles 24 toward said dirty collection system.

Said wetting system is constituted by one containment means 38, adapted to contain the cleaning liquid, one electrical pumping means 39, adapted to draw said cleaning liquid, and delivery means 40 for delivering such a liquid on the brushing means 21, 22.

Said electrical pumping means 39 is constituted for example by an electrical pump and is placed on the frame 16, in the central area of this latter, whereas said containment means 38 is constituted nu a second tank arranged in a removable manner above the front half portion of said frame 16 and the components placed in this portion of frame, and has a base that follows the outline and dimensions of the portion of frame on which it is leant and an height that doesn't interfere with the jointing element 14.

The back wall 38′ od said second tank 38 and the front wall 31′ of said first tank 31 are almost vertical and coupled to each other thereby forming a unique body 45, constituted by two above said tanks 31, 38, and having an upper surface 46 almost continuous and inclined frontally downward and side walls 47 inclined inward and upward, and finally having a vertical hole 41, preferably with a cylindrical cross-section, in correspondence of the jointing element 14, in such a way that this latter be inserted therein and be free to carry out its function, cylindrical hole 41 obtained by means of two vertical semi-cylindrical grooves identical and specular to each other, and provided on to two said vertical walls 31′, 38′ coupled to each other, respectively.

Such a body 45, along its lower base outline, is also provided with a plurality of shape recesses 48 identical and equidistant to each other, for the aim that will be described further.

Said delivery means 40 are constituted by one or more nozzles positioned in the lower part of the frame 16 and connected in a communicating manner to said pumping means 39 and turned on to the brushes 21, 22, in such a way that these latter may be wetted with the cleaning liquid drawn from the second tank 38.

Above said frame 16, a respective impact resistant means 49 is housed in correspondence of each recesses 48 of the body 45 and is protruded partially outward the body 45 with its side edge, and is adapted to absorb eventual impacts with external objects with respect the device 10, such an impact resistant means 49 is constituted for example by a roller pivoted with a vertical rotation axis, free in rotation, and made of semi-rigid elastic material, as for example rubber.

At least one electrical sanitation means 52 is fixed to the lower part of the frame 16, at the back with respect to the direction of advancement of said device 10 and to said restraining means 29, such a sanitation means 52 being constituted as for example by one or more UV lamps and being adapted to complete the sanitation of the walkable surface 11 just treated.

At least a pivoting wheel 79 is positioned in the lower part of the frame 16, at the back with respect to said electrical sanitation means 52, and is adapted to support the back part of the lower body 12, by avoiding that this latter is inclined backward.

The device 10 may be equipped with at least a detecting means 53, constituted for example by a sensor of the gyroscope type or inertial type, positioned on the frame 16 too, and adapted to detect the direction of advancement of the same device 10, that is if the operator is pushing or pulling the same device 10 for inverting the direction of advancement thereof, therefore the brushing means 21 and 22 may eventually invert the own direction of rotation and that the possible restraining means 29 positioned on the front with respect to the brushing means 21 and 22 with respect to the advancement direction of the device 10 be lifted up from the walkable surface 11, while the possible restraining means 29 positioned on the back of the brushing means 21 and 22 with respect to the advancement direction of the device 10 be lowered and put into contact with the walkable surface 11, such an operation of lifting and lowering of the possible restraining means 29 is obtained, as above described, by means the rotation of the levers 35, 36, both in a manual manner and thanks to the actuator means 51, that may be associated to the sensor means 52 by working automatically or being commanded directly by the operator by means of suitable commands.

Said movable jointing element 14, fixed vertically on the frame 16, in the barycenter point of the lower body 12, is adapted to connect the cleaning lower body 12 and the upper steering means 13 in a constraint way in rotation with respect to the vertical axis of the connecting point between the jointing element 14 and the lower body 12 and in a non-constraint way in inclination of the steering means 13 with respect to the lower body 12.

Such a jointing element 14 is constituted by at least a lengthened elastic and internally hollow push element 14 that in a rest position thereof has a rectilinear upward extension, and is constrained with its lower end portion 54 to the frame 16 and with its upper end portion 55 to the steering means 13, such a lengthened elastic element may be taken after a curvilinear extension by inclining the steering means 13, that is by moving forcefully its upper end portion 55 downward and externally and in any direction with respect to the vertical axis of the same elastic element in the rest position thereof, being able in this position with curvilinear extension to be rotate along the vertical axis of the same elastic element in the rest position thereof, without that the lower body 12 be interested by such a rotation (See FIGS. 9-12, 20).

The elastic element 14 may be curved downward up to the steering means 13 doesn't touch the walkable surface 11, so also with inclinations up to at least 90° with respect the start position thereof, allowing to the operator to insert the apparatus 10 in to space where there are present external objects 81 at the top, as for example small tables, hanging furniture, etc., without that the steering means 13 finds interferences.

By decreasing the downward push force of the upper end portion 55, the elastic element return toward the rest position, up to reach it with the removal of such a downward push force, therefore by reducing also the weight load on the operator's arms.

By applying a twisting moment to the upper end portion 55 of the lengthened elastic element 14, this latter twist along the own axis, both that it is in the position with rectilinear extension thereof and that it is in a position with a curvilinear extension thereof, by creating a twisting moment in the connecting point between its lower end portion 54 and the frame 16, by allowing to the lower body 12 to rotate on its perpendicular axis extent upward of the angle desired by the operator, without that it be inclined, so by using a limited space for change direction, and so it is advantageous for the cleaning in tight spaces. Furthermore, by rotating the lower body 12 on itself, this latter from a spaced position from the operator, it returns close to the same operator automatically, always in the starting direction of advancement (See FIGS. 13-15).

Such a lengthened elastic element 14 is constituted by a sprung element, as for example:
one spring, made of metal or polymer (shown in the attached drawings);
at least two springs inserted or co-moulded concentrically one inside the other, made of metal and/or polymer;
one metal bellow, as for example made of steel, with a core made of elastic and semi-rigid material, as for example rubber;
a pipe made of elastic and semi-rigid material, as for example rubber, provided with an internal reinforcement made of reinforcing fibers woven to each other, as for example wire made of metal, nylon, Kevlar, carbon, technopolymer;
a set of coned-disc springs joined to each other with their edges, arranged in an alternate way in succession, therefore the large base of the first coned-disc spring is joined to the large base of a subsequent second coned-disc spring, and the tight base of this latter is joined to the tight base of a subsequent third coned-disc spring, and so the remaining coned-disc springs that compose the entire lengthened elastic element 14.

Such an elastic element 14 may also be constitute by any other type of lengthened elastic and internally hollow body.

The steering means 13, lengthened upward, is constituted by a central lengthened body 56, shaped with a structure almost box-shaped provided with an external seat along one vertical front or back wall thereof, adapted to house in a removable manner one means for supplying power energy 57, as for example a rechargeable battery, adapted to power all the electrical and electronic components of the device 10, such a central lengthened body 56 being connected with its lower end portion 58 to the upper end portion 55 f the jointing element 14 and being provided at its upper end portion 59 with a shaped handle 60 and one control and settings panel 61 for the device 10.

The power supply, in addition to the means for supplying power energy 57, may be provided by the electrical domestic grid by means of electrical cables and on electrical plug (not shown) arranged on the lengthened body 56.

Such a handle 60 is of the closed ring type and is extended upward with two first lengthened segments 62 identical and specular to each other and inclined slightly outward, and continue with two second lengthened segments 63 identical e specular to each other and extended frontally, and close itself with a horizontal lengthened segment 64 which connect the upper end portion of two second segments 63.

This conformation of the handle 60 allows to the operator to grab it with one or two hands in the point that is suitable for him depending on the inclination of the steering means 13, that is this latter is basically in a vertical position thereof and the operator grabs the first two lengthened segments 62, when the steering means will be in a inclined position thereof, hence the handle 60 will be close to the walkable surface 11, the operator, for not bowing and tiring too much, will grab the third horizontal segment 64, which will be in a raised position with respect to the other segments.

The control and settings panel 61 is adapted to activate the device 10 and to select a operational program loaded in advance for the electrical and electronic component elements and means above described, and is constituted by at least a display, where the data of the device 10 are visible, and a plurality of setting means, as for example push-buttons and/or knobs and/or touch screen commands, which are built-in in the above display, furthermore, such a panel 61 is associated with at least a first electronic control and command means (not shown), constituted as for example by an electronic circuit board equipped with a microprocessor, housed inside the lengthened body 56 and powered by the battery 57.

Said first control and command means us adapted to set the activation and the power-down of the device 10 and to set the operational program loaded in advance of the electrical and electronic means comprised in the device, as for example the intensity of the operation, the direction of the rotation of the brushing means 21 and 22, etc.

Said first control and command means may be also associated to at least a second electronic control and command means (not shown), constituted as for example by an electronic circuit board equipped with at least a microprocessor too, and positioned on the frame 16, which second control and command means is adapted to receive instructions from the first control and command means and to adjust the activation and the shot-down of the electric and electronic means comprised in the device, to carry out the operational program and to adjust the intensity of operation.

Said first control and command means is also associated to the sensor means 53, for being able to carry out the above the described operations, particularly, said first control and command means receives the data from the sensor means 53 and sends command to the second control and command means.

Said first control and command means is also associated to at least an additional detecting means (not shown) positioned in the upper end portion 59 of the steering means and adapted to detect the presence of the operator in close proximity to the device 10, in such a way that if the device 10 is in the operational state and the operator is not detected, said first control and command means sends the command to the second electronic control and command means to deactivate all the electrical and electronic means, which are operative in that moment, for then re-activate them when the operator is detected.

Such an additional detecting means is constitute by at least a proximity sensor, that may be of the ultrasound type or of the optical type and position in the area of the control and setting panel 61, thereby be able to detect the presence of the operator within a determined distance, and/or may be of the capacitive type and position on the handle 60, thereby be able to detect if the operator is grabbing or not said handle 60 with at least one hand.

Said box-shaped structure of the lengthened body 56 is provided inside with a lengthened fixing means 66 (See FIGS. 16-17), constituted by a vertical rod 71 to which an horizontal rod 68 is fixed on its the upper part and housed in a sliding manner within a vertical hollow guide member 67, and moved by the operator by means of the horizontal rod 68, which is extended with at least one its side end portion beyond the side vertical walls of the body 56, by means of two vertical through slots 70 provided on to this latter and specular to each other.

In the starting position of the device 10, such a fixing means 66 from a rest raised position thereof, in which it is maintained by means of fixing means of the magnetic type or by means of a pawl wherein it is engaged the horizontal rod 68, may slide downward by the operator in an operational lowered position thereof passing through the opened end portion 58 and stopping at the internal base of the jointing means 14, where an end-of-stroke element 72 is positioned, constituted preferably by a vertical hollow cylinder into which the lower end portion 73 of the same fixing means 66 engage itself, in this manner the device 10 is locked in the starting position thereof, for be able to be stored, therefor by avoiding the possibility that the jointing means 14 may be curved.

A plurality of wheels 74 may be fixed under the frame 16 in a raised manner, preferably in front with respect to the brushing means 21 and 22, in such a way that when the fixing means 66 is in the operational position thereof, the operator may incline the device 10 by leant such wheels 74 on the walkable surface 11, therefore it is possible to easily transport it without that the brushes 21 and 22 and the restraining means 29 create friction, since they are in a raised position thereof.

Said lengthened body 56 may be connected with its lower end portion 58 to the jointing element 14 by means of a joining means 75, which is lockable in position and adapted to maintain the steering means 13 in an operational position thereof or to release it from such a position for allowing it to be inclined without to intervene on the jointing element 14 (See FIGS. 18, 19), therefore for being able to reduce the overall dimensions and packaging or storing it easily.

Such a joining means 75 is constituted by a lower part 76 connected by means of a horizontal pivot to an upper part 77, in such a way that two parts are tilting to each other, and may be locked in position, when such two parts 76 and 77 are adjacent one above the other, by means of a locking means 78, constituted as for example by a bolt.

The lower part 76 is fixed to the upper end portion of the jointing element 14, whereas the upper part 77 is fixed to the lower end portion 58 of the lengthened body 56 of the steering means 13. Said parts 76 and 77 are each provided with a respective vertical concentric through hole 79, 80 (See FIG. 19), adapted to allow the passage of the vertical rod 71 of the fixing means 66.

In addition to the aims and advantageous above listed, such a device has the advantageous characteristic that by using a jointing means 14, that is of the elastic type, the vibrations created by the device 10 during its operational state, or possible impacts with external objects, are absorbed by the same jointing means 14 that twist automatically for return then in the position thereof in which it was before the impact, without so weigh on the operator, therefore who may work with small loss of energy and with few traumas.

1. A scrubber-dryer device for cleaning walkable surfaces, adapted to move forward automatically and to be steered manually by an operator, and comprising: a cleaning lower base body connected on its upper part by means of a movable jointing element to a steering means lengthened upward, said lower base body comprising a frame to which one or more lower brushing means are fixed to and actuated in rotation by means of respective electrical actuating means, and adapted to brush the walkable surfaces by removing dirt from and to create a friction with the walkable surface such to move forward automatically in a rectilinear way the device, at least a wetting system fixed to the frame, and positioned in correspondence of said brushing means and adapted to wet them, and at least a dirt collection system fixed to the frame, positioned on the back of said brushing means with respect the travel direction of the device and adapted to collecting the dirt brushed by the same brushing means, said wetting system comprising one containment means adapted to contain the cleaning liquid, one electrical pumping means adapted to draw the cleaning liquid, and delivery means for delivering such a liquid, said dirt collection system comprising at least one restraining means adapted to restrain the dirt conveyed by the brushing means, at least one suction means adapted to suck the restrained dirt, and at least a collection means adapted to contain the sucked dirt, said steering means comprising an upward lengthened body joined at the top thereof to a handle and being of the inclinable type in any direction from a starting position perpendicular with respect to the lower body and being adapted to steer the device, wherein said jointing element comprises a lengthened elastic and internally hollow push element constrained with its lower end portion to the frame in the barycenter point of the lower body and with its upper end portion to the steering means, and that from the rest position thereof, in which it has a rectilinear upward extension, it may be curved downward by applying a downward and outward push force to the upper end portion thereof and in any direction with respect to its vertical axis when it is in the rest position thereof, therefore to incline downward the steering means, and in this curved position being able to rotate along the vertical axis of the rest position of the same elastic element, without that the lower body is interested by such a rotation, by decreasing the downward push force of the upper end portion, the elastic element returning automatically toward the rest position thereof, such an elastic element being also adapted to receive a twisting moment to its upper end portion by means of said steering means for then applying said twisting moment, with its lower end portion to the frame of the lower body, therefore this latter rotates on its perpendicular axis extent upward of the angle desired by the operator, without be inclined.

2. The device according to claim 1, wherein such a lengthened elastic and internally hollow push element comprises a sprung element, made of metal or polymer.

3. The device according to claim 2, wherein said sprung element comprises at least two springs inserted or co-moulded concentrically one inside the other, made of metal and/or polymer.

4. The device according to claim 2, wherein said sprung element comprises one metal bellow, with a core made of elastic and semi-rigid material.

5. The device according to claim 2, wherein said sprung element comprises a pipe made of elastic and semi-rigid material, provided with an internal reinforcement made of reinforcing fibers woven to each other.

6. The device according to claim 2, characterized in that said sprung element comprises a set of coned-disc springs joined to each other with their edges, arranged in an alternate way in succession, therefore the large base of the first coned-disc spring is joined to the large base of a subsequent second coned-disc spring, and the tight base of this latter is joined to the tight base of a subsequent third coned-disc spring, and so the remaining coned-disc springs that compose the entire lengthened elastic element (14).

7. The device according to claim 1, wherein said electrical actuating means, comprised by electrical motors, are arranged on the upper part of said frame and above to the respective brushing means that they have to actuate, said brushing means being arranged side by side to each other in a transversal manner with respect the orientation of the lower body, and rotating in opposite directions to each other along an axis which is slightly inclined inward with respect to the vertical of the device leant on a horizontal plane.

8. The device according to claim 1, wherein said steeling means comprises at the top thereof one control and settings panel associated with at least a first electronic control and command means for activating the device and to select an operational program, loaded in advance in the first electronic control and command means, of the electrical and electronic component elements of the same device, said control and setting panel comprising at least a display, where the data of the device are visible, and a plurality of setting means, which are built-in in the above display.

9. The device (10) according to claim 8, wherein said first control and command means is associated to at least a second electronic control and command means, comprising an electronic circuit board equipped with at least a microprocessor too, and positioned on the frame, which second control and command means being adapted to receive instructions from the first control and command means and to adjust the activation and the shot-down of the electric and electronic means comprised in the device and to carry out the selected operational program and to adjust the intensity of operation.

10. The device according to claim 1, wherein said handle is of the closed ring type and is extended upward with two first lengthened segments identical and specular to each other and inclined slightly outward, and continue with two second lengthened segments identical e specular to each other and extended frontally, and close itself with a horizontal lengthened segment which connect the upper portion of two second segments.

11. The device according to claim 1, wherein said restraining means for the dirt comprises a lengthened semi-circular support provided with at least a vertical through hole and which supports on its lower edge at least a lengthened element made of a semi-rigid material, said support being positioned transversally with respect to the orientation of the device with its concave part turned toward the two brushing means and being connected to the lower part of the frame by means of at least two pair of levers pivoted at their end portions parallel to each other, and arranged longitudinally with respect to the orientation of the device, which levers being adapted to rotate on their end portions according the vertical movement caused by the restraining means that follows the conformation of the walkable surface, or by being lifted or lowered manually or automatically when the operator considers it necessary, said restraining means being kept lifted by means of quick fixing means or magnetic means or electromagnetic means.

12. The device according to claim 11, wherein said levers are made of elastic metal and are structured and adapted to be bent transversally when the restraining means bumps laterally against an external obstacle for then returning automatically in the original position thereof after having passed such an external obstacle.

13. The device according to claim 11, wherein said levers are rotated vertically by means of one electrical actuator means placed on the upper part of the frame.

14. The device according to claim 1, wherein said restraining means are provided with two support wheels, placed laterally and/or centrally with respect to the lengthened support and are fixed by means of metallic brackets.

15. The device according to claim 11, wherein said suction means is of the electrical type, and is arranged on the upper part of said frame and above said restraining means, and with its inlet mouth is connected in a communicating way to said vertical through hole, and with its outlet mouth is connected in a communicating manner to said collection means.

16. The device according to claim 15, wherein said collection means comprises a first tank arranged in a removable manner above the back half portion of said frame and the components placed in this portion of frame, and has a base that follows the outline and the dimensions of the portion of frame on which is leant and a height that doesn't interfere with the jointing element.

17. The device (10) according to claim 1, wherein said electrical pumping means, (39 comprising one electrical pump, is placed on the frame in the central area of this latter, and is associated in a communicating manner with said containment means, comprising a second tank arranged in a removable manner above the front half portion of said frame and the components placed in this portion of frame, and has a base that follows the outline and dimensions of the portion of frame on which it is leant and an height that doesn't interfere with the jointing element.

18. The device according to claim 15, wherein the first tank and the second tank are joined to each other and form a unique body having an upper surface almost continuous and inclined frontally downward and side walls inclined inward and upward, and finally having a vertical hole, in correspondence of the jointing element, in such a way to be inserted therein and be free to carry out its function.

19. The device according to claim 18, wherein said body, along its lower base outline, is also provided with a plurality of shape recesses identical and equidistant to each other, in correspondence of each of which, above said frame, a respective impact resistant means is housed and protruded partially outward the device, such an impact resistant means comprises a roller pivoted with a vertical rotation axis, free in rotation, and made of semi-rigid elastic material.

20. The device according to claim 1, wherein at least one electrical sanitation means is fixed to the lower part of the frame at the back of said restraining means with respect to the direction of advancement of said device, such a sanitation means comprising one or more UV lamps and being adapted to complete the sanitation of the walkable surface just treated, and at least a pivoting wheel being positioned in the lower part of the frame at the back with respect to said electrical sanitation means and being adapted to support the back part of the lower body by avoiding that this latter is inclined backward.

21. The device according to claim 8, wherein said first electronic control and command means is also associated to at least an additional first detecting means positioned on the upper part of the steering means and adapted to detect the presence of the operator in close proximity to the device in such a way that if the device is in the operational state thereof and the operator is not detected, said first electronic control and command means sends the command to the second electronic control and command means to deactivate all the electrical and electronic means, which are operative in that moment, for then re-activating them when the operator will be detected.

22. The device according to claim 21, wherein said first detecting means comprises a proximity sensor of the ultrasound type or of the optical type and is positioned in the area of the control and setting panel.

23. The device according to claim 21, wherein said first detecting means comprises a proximity sensor of the capacitive type positioned on the handle.

24. The device according to claim 1, wherein said first control and command means is also associated to the sensor means comprising a sensor of the gyroscope type or inertial type, positioned on the frame, and adapted to detect the direction of advancement of the device when the operator is pushing or pulling the same device for inverting the direction of advancement thereof, therefore the ⁻brushing means may eventually invert the own direction of rotation and that the possible restraining means positioned on the front with respect to the brushing means with respect to the advancement direction of the device be lifted up from the walkable surface.

25. The device according to claim 1, wherein said box-shaped structure of the lengthened body is provided inside with a lengthened fixing means, which may slide vertically from a raised rest position to an lowered operational position thereof, and vice versa, for locking or unlocking the device in the starting position thereof

26. The device according to claim 25, wherein said lengthened fixing means comprises a vertical rod to which an horizontal rod is fixed on its the upper part and housed in a sliding manner within a vertical hollow guide member and moved by the operator by means of the horizontal rod which is extended with at least one its side end portion beyond the side vertical walls of the body by means of two vertical through slots provided on to this latter and specular to each other, said lengthened fixing means being maintained in the raised rest position thereof by means of fixing means of the magnetic type or by means of a pawl wherein it is engaged the horizontal rod, said lengthened fixing means, in the operational lowered position thereof, is engaged with its lower end portion into an end-of-stroke element positioned inside the base of the jointing element.

27. The device according to claim 26, wherein a plurality of wheels is fixed under the frame in a raised manner, in such a way that when the fixing means is in the operational position thereof, the operator may incline the device by leant such wheels on the walkable surface, to transport it with the brushes and the restraining means that are in a raised position thereof.

28. The device (10) according to claim 1, wherein said lengthened body is connected with its lower end portion to the upper end portion of the jointing element by means of a joining means, which is lockable in position and adapted to maintain the steering means in an operational position thereof or to release it from such a position for allowing it to be inclined without to intervene on the jointing element, in a way to reduce the overall dimensions and packaging or storing it, such a joining means comprising a lower part connected in a tilting manner to an upper part (77), that and may be locked in adjacent position, one above the other, by means of a locking means.

29. The device according to claim 28, wherein said parts each provided with a respective vertical concentric through hole adapted to allow the passage of the vertical rod of the fixing means.

30. The device according to claim 1, wherein said lengthened body is provided with an external seat along one vertical front or back wall thereof, adapted to house in a removable manner one means for supplying power energy adapted to power all the electrical and electronic components of the device.
